(19) United States
(12) Patent Application Publication
(10) Pub. No.: US 2021/0220286 A1
(43) Pub. Date: Jul. 22, 2021

(54) PHARMACEUTICAL COMPOSITION COMPRISING AMORPHOUS SUNITINIB

(71) Applicant: SYNTHON B.V., Nijmegen (NL)

(72) Inventors: Sonia GARCIA JIMENEZ, Sant Boi de Llobregat (ES); Luis NOGUEIRAS NIETO, Sant Boi de Llobregat (ES); Lisardo ALVAREZ FERNANDEZ, Sant Boi de Llobregat (ES); Rohit KUMAR, Sant Boi de Llobregat (ES)

(21) Appl. No.: 17/050,696

(22) PCT Filed: Apr. 25, 2019
(86) PCT No.: PCT/EP2019/060682

§ 371 Date: Oct. 26, 2020

(30) Foreign Application Priority Data
Apr. 26, 2018 (EP) 18169549.5

(51) Int. Cl. A61K 9/48; A61K 9/20; A61K 31/404; A61J 1/03

(52) U.S. Cl. CPC A61K 9/4866; A61K 9/2027; A61J 1/035; A61K 31/404; A61K 9/2054

(57) ABSTRACT

The present invention relates to a pharmaceutical composition comprising a solid dispersion of sunitinib L-malate and polyvinylpyrrolidone in a primary packaging comprising means to absorb water. The invention further relates to the use of said composition as a medicament, particularly in the treatment of a tyrosine kinase-related disorder.

### BACKGROUND OF THE PRESENT INVENTION

Sunitinib, chemically (Z)—N-[2-(diethylamino)ethyl]-5-(5-fluoro-2-oxo-2,3-dihydro-1H-indol-3-ylidenemethyl)-2,4-dimethyl-1H-pyrrole-3-carboxamide of formula (I),

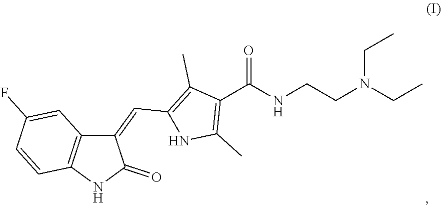 [SMILES: CCN(CC)CCNC(=O)c1c(C)[nH]c(/C=C2\C(=O)Nc3ccc(F)cc32)c1C]

is a pharmaceutically active compound used for the treatment of gastrointestinal stromal tumors (GIST), metastatic renal cell carcinoma (MRCC) and pancreatic neuroendocrine tumors (pNET). The compound was discovered by Sugen and is disclosed in WO0160814. The compound may form acid addition salts, e.g. sunitinib L-malate, which is the active ingredient in the medicinal product sold under the brand name Sutent® by Pfizer. Sunitinib L-malate exhibits polymorphism. WO03016305 discloses form I and II of sunitinib L-malate, processes for their preparation and compositions comprising these forms. Form I is the most stable form and is present in the marketed capsules. Form II is more soluble, but hygroscopic and less stable. Other polymorphic forms of sunitinib L-malate are disclosed in WO2009067686, WO2009104021, WO2010010454, WO2010055082, WO2010076805, WO2011092664 and CN104693187.

Amorphous drugs show in general better solubility and bioavailability than the crystalline forms. It might therefore be an advantage to develop a pharmaceutical composition comprising amorphous sunitinib L-malate.

WO2009156837 discloses amorphous sunitinib L-malate. Amorphous compositions comprising sunitinib and its acid addition salt are disclosed in WO2009100929 and WO2010039798. WO2010039798 (paragraph [0045]) mentions that many experiments performed by the inventors attempting to prepare amorphous sunitinib malate were unsuccessful, i.e., the sunitinib L-malate obtained was not amorphous. In our laboratory, it was confirmed that sunitinib L-malate crystallizes very easily.

WO2009100929, WO2010039798, WO2013160916 and CN106974890 disclose ways to stabilize amorphous sunitinib L-malate by preparing solid dispersions with a polymer. In addition, WO2009100929 provides capsule formulations comprising the solid dispersions disclosed. There is hardly any data available on the stability upon storage of solid dispersions of sunitinib L-malate or finished dosage forms comprising the solid dispersions. In our laboratory it was observed that many of the solid dispersions comprising sunitinib L-malate suffer, upon storage, from the formation of significant amount of impurities.

Thus in view of the prior art cited above, there is still a need for stable, amorphous compositions comprising sunitinib L-malate that do fulfill the required purity levels as demanded for pharmaceutical products, even after long term storage.

### BRIEF DESCRIPTION OF THE PRESENT INVENTION

The present invention provides a pharmaceutical composition comprising a solid dispersion of sunitinib L-malate and polyvinylpyrrolidone in a primary packaging comprising means to absorb water.

It also provides a process for preparing said pharmaceutical composition comprising wet granulation.

Said pharmaceutical composition may be used as a medicament, particularly in the treatment of tyrosine kinase-related disorder.

### DETAILED DESCRIPTION OF THE PRESENT INVENTION

Sutent® capsules contain sunitinib L-malate form I. This polymorphic form is the most stable form at ambient conditions. Sunitinib L-malate form II is more soluble, but less stable and hygroscopic. Other forms of sunitinib L-malate have been disclosed in the prior art.

Since amorphous drugs are in general more soluble and exhibit better bioavailability than the crystalline forms, it would be desirable to have sunitinib L-malate in amorphous form. However, drugs that can exist in either amorphous or crystalline form tend to crystallize over time when present in amorphous state because the crystalline form of the drug is a lower-energy state than the amorphous form.

One of the most successful strategies to stabilize the amorphous state of a compound is the preparation of a solid dispersion. The term solid dispersion has been defined as a dispersion of one or more Active Pharmaceutical Ingredients (APIs) in an inert carrier or matrix at the solid state, prepared by a solvent or melting process or a combination of the two. Depending on the physical state of the carrier, which is crystalline or amorphous, the solid dispersions are divided into crystalline solid dispersions and amorphous solid dispersions respectively. Amorphous carriers used are mostly polymers. In amorphous solid dispersions, the API is dispersed in very small size and exists in supersaturated state in amorphous carriers because of forced degradation. The amorphous carriers can increase the wettability and dispersibility of drugs as well as inhibit the precipitation process of drugs when amorphous solid dispersions are dissolved in water. These properties along with the fast dissolution rate of amorphous carriers due to the low thermodynamic stability of amorphous state carriers enhance the drug solubility and release rate. Despite the high active research interests, the number of marketed products arising from solid dispersion approaches is still low. This low number is mainly due to scale-up problems and physicochemical instability in the manufacturing process or during storage leading to phase separation and crystallization (Vo et. al, Eur. J. Pharm. Biopharm., 85 (2013) 799-813).

It is not self-evident that a given drug will form an amorphous solid dispersion with just any polymer, and that, even in the event the solid dispersion is formed, it will be stable over time. Factors playing a role herein are the physicochemical properties of both API and polymer, the ratio of API to polymer used and the technique used to prepare the solid dispersion. Techniques to prepare solid dispersions often require very specific conditions for each combination of API and polymer.

The process selected to prepare the pharmaceutical compositions of the present invention is the solvent evaporation method, because in this method problems related to decomposition, as observed when applying the melting method, are prevented. An important prerequisite of the solvent evaporation method is the sufficient solubility of the drug and the carrier in the solvent system. Finding a suitable non-toxic solvent is sometimes difficult because carriers are hydrophilic whereas drugs may tend to be hydrophobic.

In order to obtain pharmaceutical compositions comprising a solid dispersion of sunitinib L-malate and a polymer, exhibiting adequate dissolution and excellent long term stability, which are suitable for production on commercial scale, the use of several, commonly used, polymers has been studied. The polymers investigated are polyvinyl-pyrrolidone, copovidone, hydroxypropyl cellulose, hydroxypropyl methylcellulose, PVA, polyethylene glycol, poloxamer and Eudragit E-100.

Most polymers studied were found to be unsuitable to be used in accordance with the present invention for various reasons. Some of the polymers did not result in fully amorphous solid dispersions, while other polymers did give rise to amorphous solid dispersions wherein sunitinib L-malate started to crystallize over time during storage. Moreover, some polymers were found to be unsuitable to be used in accordance with the present invention due to their solubility/gelling behavior in the solvent system.

It was found that polyvinylpyrrolidone, also known as PVP or povidone, is a particularly preferred polymer. Use of this polymer results in solid dispersions wherein sunitinib L-malate is present in fully amorphous form and wherein, even after storage under stressed conditions, no conversion into crystalline material occurs. Depending on the degree of polymerization, PVP with various molecular weights can be obtained. The person skilled in the art will be able to select the grade of PVP with a specific molecular weight to be used in accordance with the present invention. PVP grades with molecular weights of 20.000 to 100.000 are particularly preferred. Even more preferred to be used in accordance with the present invention is PVP with a molecular weight ranging from 30.000 to 60.000.

The present invention provides a pharmaceutical composition comprising a solid dispersion of sunitinib L-malate and polyvinylpyrrolidone in a primary packaging comprising means to absorb water.

We experienced that the pharmaceutical compositions comprising a solid dispersion of sunitinib L-malate suffer, upon storage, from the formation of impurities and do not reach the required purity levels as demanded for pharmaceutical products. Several degradation impurities are formed upon storage by e.g. hydrolysis and oxidation. In our laboratory, different strategies to decrease and control the level of the individual and total amount of impurities formed during storage have been studied.

The influence of blister pack materials having different WVTR's on the impurity profile of the pharmaceutical compositions upon storage at 40° C./75% RH was studied. Blisters belong to the group of primary packaging material in pharmaceutical industry. The water vapor transmission rate (WVTR), also moisture vapor transmission rate (MVTR), is a measure of the passage of water vapor through a substance. Various techniques are available to measure WVTR and several standard methods are described in e.g. ISO, ASTM, BS and DIN, like ASTM F1249 and DIN53122. The conditions under which the measurement is made has a considerable influence on the result. Both the temperature and the humidity gradient across the sample need to be measured, controlled and recorded with the result. Three different blister pack materials with films having different WVTR's were studied. Triplex, a PVC/PE/PVDC film, has a WVTR value of 0.15 g/m²/day at 40° C./75% RH, while Aclar, which is a PTFE film, has a WVTR value of 0.07 g/m²/day at 40° C./75% RH. Cold Form Foil (CFF), also known as Alu-Alu foil, has a WVTR value of ≤0.01 g/m²/day at 23° C./100% RH. It was noted that the amount of impurities in the composition after 3 months storage at 40° C./75% RH decreased with decreasing WVTR value. Although Alu-Alu blister packs gave the best results, the required purity levels as demanded for this pharmaceutical product is not reached.

The use of several types of antioxidants like vitamin C, vitamin E, butylated hydroxytoluene (BHT) as additive to the pharmaceutical composition and their effect on the impurity profile was studied. Surprisingly enough, instead of achieving a decrease in the level of (oxidation) impurities, in most cases the use of antioxidants led even to an increase in impurities in the compositions after storage at 40° C. and 75% RH when compared to the composition lacking antioxidant.

Polyvinylpyrrolidone (PVP) is the polymer used in accordance with the present invention to form a solid dispersion with sunitinib L-malate. It is known that PVP contains peroxides and these components may promote oxidative degradation. Therefore, two different grades of PVP with different content of peroxides were used to prepare the solid dispersions of the present invention. The first type of PVP used, contains ≤400 ppm of peroxides. Plasdone™ K29/32 is a typical example of such grade of PVP. The peroxide content of the second type of PVP used is significantly lower: ≤150 ppm. A typical example of such low peroxide content grade of PVP is Kollidon® 30 LP. Both polymers were used to prepare solid dispersions with sunitinib L-malate which were further processed into the pharmaceutical compositions in accordance with the present invention. The impurity profiles after storage at 40° C./75% RH in bottles and blisters seem to indicate that only slightly better results are obtained with the low peroxide content PVP.

The influence of different capsule shells, gelatin and HPMC, on the level of impurities of the pharmaceutical composition was studied. Colored gelatin capsule shells do contain several metal oxides and exhibit relatively high moisture content with a LOD of 13-16% when compared to HPMC transparent capsule shells that are free of metal oxides and exhibit a LOD of ≤9%. The expected effect of these differences in capsules on the level of impurities of the compositions after storage at 40° C./75% RH was completely absent. No significant differences were found.

Since magnesium may catalyze reactions leading to the formation of degradation impurities, the commonly used lubricant magnesium stearate was replaced with sodium stearyl fumarate and stearic acid in order to investigate the effect of the different type of lubricants on the impurity profile of the composition. No significant improvement on the level of impurities after storage of the compositions at 50° C. was observed for the two magnesium-free lubricants over magnesium stearate.

In order to study the effect of headspace volume on the impurity profile of the pharmaceutical compositions upon storage at 40° C./75% RH, the compositions were stored in different sizes of bottles. No significant differences in impurity profiles were found between compositions stored in large volume (75 ml) bottles and compositions stored in small volume (30 ml) bottles, indicating that the size of the headspace and the humidity/oxygen present in this space plays hardly any role on the stability of the compositions.

It can be concluded that none of the commonly applied methods available to the person skilled in the art to reduce the amount of degradation impurities formed e.g. hydrolysis and oxidation did result in compositions comprising a solid dispersion of sunitinib L-malate and polyvinylpyrrolidone displaying acceptable purity levels upon long term storage. Very surprisingly it was found that formation of impurities in the pharmaceutical composition comprising a solid dispersion of sunitinib L-malate and polyvinylpyrrolidone can be very effectively suppressed by using primary packaging comprising means to absorb water. In this way, compositions with purity levels as demanded for pharmaceutical products are obtained.

Primary packaging materials are those that are in direct contact with the product. Typical examples are blisters and bottles. These primary packaging materials may comprise means to absorb water. This can be achieved by e.g. inclusion of a desiccant.

In a preferred embodiment of the invention, the primary packaging comprising means to absorb water is a blister pack having a water absorption capacity of at least 2.0 g/m² at 40° C./90% RH. The water absorption capacity is determined by gravimetric measurement over 24 hours. More preferably, the primary packaging material is a blister pack having a water absorption capacity of at least 3.5 g/m² at 40° C./90% RH. Even more preferably, the primary packaging material is a blister pack having a water absorption capacity of at least 5 g/m² at 40° C./90% RH. Most preferably, the primary packaging material is a blister pack having a water absorption capacity of at least 6.1 g/m² at 40° C./90% RH. A laminated cold forming blister pack comprising desiccant is a specifically preferred blister pack to be used in accordance with the present invention. Typical examples of commercially available desiccated Alu-Alu blisters are known under the name Dessiflex™. Several grades are available with different water absorption capacities. Dessiflex™ 2.0 is for example a laminated cold forming blister pack comprising desiccant having a guaranteed water absorption capacity of at least 2.0 g/m² at 40° C./90% RH, while Dessiflex™ Plus and Dessiflex™ Ultra are dessicated Alu-Alu blisters having a water absorption capacity of at least 3.6 and 6.1 g/m² at 40° C./90% RH respectively.

In another embodiment of the present invention, the primary packaging comprising means to absorb water is a capped bottle having a water absorption capacity of at least 5.3 g/dm³ at 23° C./40% RH. More preferably, the water absorption capacity of the capped bottle is at least 10 g/dm³ at 23° C./40% RH. Most preferably, the water absorption capacity of the capped bottle is at least 15 g/dm³ at 23° C./40% RH. Examples of such capped bottles comprising means to absorb water are bottles having a cap containing desiccant, e.g. silica gel. Typical examples of commercially available caps containing silica gel are the Duma® Twist-Off Caps wherein different quantities of silica gel are integrated, resulting in varying water absorption capacity values. In a specifically preferred embodiment, the capped bottle is a HDPE bottle and the cap comprises desiccant.

At least a major portion of sunitinib L-malate in the pharmaceutical composition is amorphous. The term “a major portion” of sunitinib L-malate means that at least 60% of the drug is in amorphous form, rather than a crystalline form. Preferably, sunitinib L-malate in the pharmaceutical composition is at least 80% in amorphous form. More preferably, sunitinib L-malate in the composition is “almost completely amorphous” meaning that the amount of sunitinib L-malate in the amorphous form is at least 90% as measured by powder X-ray diffraction or any other standard quantitative measurement. Most preferably, sunitinib L-malate in the pharmaceutical composition is in a completely amorphous form within the detection limits of the techniques used for characterization.

The weight ratio of sunitinib L-malate to polyvinylpyrrolidone in the solid dispersion ranges from 1:1 to 1:2. At lower ratios of sunitinib L-malate to pyrrolidone, e.g. 1:0.5, amorphous product is obtained, but upon storage conversion into crystalline sunitinib L-malate occurs. Ratios of sunitinib L-malate to polyvinylpyrrolidone above 1:2 will provide stable amorphous compositions, but are accompanied with an unacceptable increase in capsule size.

The pharmaceutical composition of the present invention is very suitable for production on commercial scale. The solid dispersion comprising sunitinib L-malate is obtained by applying the process of wet granulation. This conventional technique can be carried out with equipment commonly used in pharmaceutical industry, which is a big advantage over other known techniques to prepare solid dispersions, like hot melt extrusion, freeze drying and spray-drying. These techniques do require specific, expensive equipment.

The pharmaceutical composition of the present invention is obtained by dissolving, in the first step of the process, sunitinib L-malate and polyvinylpyrrolidone in a solvent mixture. The non-toxic solvent mixture in accordance with the present invention is an aqueous acidic solution. Preferably, the solvent mixture is a solution of water comprising a mineral acid. More preferably, the mineral acid is hydrochloric acid. Even more preferably, a solution comprising of a mixture of water and 1N hydrochloric acid is used. The amount of acid present in the solvent mixture may range. Most preferably, the solvent system used in accordance with the present invention is consisting of water and 1N hydrochloric acid in a ratio of 81.5:18.5 (w/w). Both sunitinib L-malate and polyvinylpyrrolidone dissolve well in the solvent mixture upon heating at a temperature ranging from 35 to 85° C. At low temperatures, the viscosity of polyvinylpyrrolidone in the solvent mixture is high, making it difficult to process the solution, while at high temperatures the amount of impurities increases. Preferably, the temperature of the solvent system is ranging from 45 to 55° C.

After the first step of dissolving sunitinib L-malate and polyvinylpyrrolidone in the solvent mixture, the resulting solution is mixed with a diluent. The diluent to be used in accordance with the present invention may be any diluent known to a person of ordinary skill in the art. Particularly, the diluent to be used in accordance with the present invention is an inorganic diluent, polysaccharide, mono- or disaccharide or sugar alcohol. Microcrystalline cellulose is a particularly preferred diluent.

After the step of mixing the solution, comprising sunitinib L-malate and polyvinylpyrrolidone in the solvent mixture, with a diluent, the solvent is evaporated. The evaporation is carried out by techniques known to a person of ordinary skill in the art.

In a particularly preferred embodiment, the solution, comprising sunitinib L-malate and polyvinylpyrrolidone in the solvent mixture, is sprayed over the diluent in a fluid bed reactor and the resulting mixture is subsequently dried. Preferably, the solvent mixture used is an aqueous acidic solvent system and the diluent used is microcrystalline cellulose.

The resulting blend is then mixed with further excipients. The pharmaceutical composition of the present invention comprising the solid dispersion of sunitinib L-malate and polyvinylpyrrolidone, further comprises, besides the diluent in the intragranular phase, one or more extragranular pharmaceutically acceptable excipients. The excipients to be used in accordance with the present invention are well-known and are those excipients which are conventionally used by the person skilled in the art. Depending on the dosage form chosen for the pharmaceutical composition, the person skilled in the art will be able to select suitable pharmaceutically acceptable excipients. Preferably, the dosage form is an immediate release hard shell capsule and the pharmaceutically acceptable excipients are chosen from one or more diluents, disintegrants or lubricants.

The diluent to be used in accordance with the present invention may be any diluent known to a person of ordinary skill in the art. Particularly, the diluent to be used in accordance with the present invention is an inorganic diluent, polysaccharide, mono- or disaccharide or sugar alcohol. Microcrystalline cellulose is a particularly preferred diluent.

The disintegrant to be used in accordance with the present invention may be any disintegrant known to a person of ordinary skill in the art. Suitable disintegrants to be used in accordance with the present invention are selected from the group consisting of croscarmellose sodium, crospovidone or sodium starch glycolate. Croscarmellose sodium is a particularly preferred disintegrant.

The lubricant to be used in accordance with the present invention may be any lubricant known to a person of ordinary skill in the art. Magnesium stearate is a particularly preferred lubricant.

In a particularly preferred embodiment of the present invention, microcrystalline cellulose, croscarmellose sodium and magnesium stearate are present as extragranular excipients.

After mixing the blend, comprising the solid dispersion of sunitinib L-malate and polyvinylpyrrolidone, with further excipients, the final blend is compressed into tablets, or filled into capsules, using equipment and methods well-known in the art.

The pharmaceutical composition of the present invention exhibits excellent long term stability. Even after 6 months at 40° C./75% RH, no conversion into any crystalline form of sunitinib L-malate was observed. Moreover, the composition of the present invention fulfills the required purity levels as demanded for pharmaceutical products even after long term storage under accelerated conditions.

The pharmaceutical composition of the present invention displays dissolution behavior typical for immediate-release formulations. The composition of the present invention exhibits a dissolution rate of at least 85% in 15 minutes when tested in 900 ml 0.1 N hydrochloric acid pH 1.0 at 37° C., 75 rpm in a USP apparatus II.

The pharmaceutical composition in accordance with the present invention may be used as a medicament. The pharmaceutical composition typically may be used in the treatment of a tyrosine kinase-related disorder, preferably for the treatment of gastrointestinal stromal tumors (GIST), metastatic renal cell carcinoma (MRCC) and pancreatic neuroendocrine tumors (pNET).

The present invention is illustrated by the following Examples.

### EXAMPLES

### Example 1: Preparation of Capsule Compositions Comprising a Solid Dispersion of Sunitinib L-Malate and PVP in Different Ratios (1:1, 1:1.5, 1:2)

| | | | |
|---|---|---|---|
| Component | Ratio 1:1 | Ratio 1:1.5 | Ratio 1:2 |

Intragranular components

| | | | | | | |
|---|---|---|---|---|---|---|
| Sunitinib L-malate | 66.83 | 27.84 | 66.83 | 24.75 | 66.83 | 21.56 |
| PVP | 66.83 | 27.84 | 100.24 | 37.13 | 133.66 | 43.11 |
| MCC | 30.49 | 12.70 | 25.93 | 9.60 | 28.21 | 9.10 |
| Purified water:HCl 1N | | q.s. | | q.s. | | q.s. |
| 81.5:18.5 (w/w) | | | | | | |

Extragranular components

| | | | | | | |
|---|---|---|---|---|---|---|
| MCC | 67.45 | 28.11 | 67.55 | 25.02 | 70.45 | 22.73 |
| Croscarmellose sodium | 7.20 | 3.00 | 8.10 | 3.00 | 9.30 | 3.00 |
| Magnesium stearate | 1.20 | 0.50 | 1.35 | 0.50 | 1.55 | 0.50 |
| Capsule content weight | 240.00 | 100.00 | 270.00 | 100.00 | 310.00 | 100.00 |

Polyvinylpyrrolidone and sunitinib L-malate were dissolved in a solution of hydro-chloric acid in water under heating and stirring. The solution was sprayed and dried under heating and stirring over the sieved microcrystalline cellulose (intragranular part) in a fluid bed reactor. The obtained granules were milled. Microcrystalline cellulose (extragranular part) and croscarmellose sodium were sieved to deagglomerate and mixed with the sieved granulate in a tumbling mixer. Magnesium stearate was sieved and added to the tumbling mixer and the resulting mixture was mixed. The homogeneous blend was encapsulated using a dosator filling machine into capsules.

The capsules were packed in suitable packaging material.

### Example 2: Stability Results for 50 mg Capsules with Different Ratios of Sunitinib L-Malate to PVP (1:1, 1:1.5, 1:2)

The capsules have the compositions as given in example 1 and are prepared by the process given in that example.

| | | | 40° C./75% RH | | | | | |
|---|---|---|---|---|---|---|---|---|
| | | | 1 month | | 3 months | | 6 months | |
| Ratio | Impurities | t = 0 | Bottle¹ | Blister² | Bottle¹ | Blister² | Bottle¹ | Blister² |

Main degradation impurities - UHPLC (%)

| | | | | | | | | |
|---|---|---|---|---|---|---|---|---|
| 1:1 | RRT 1.07 | 0.09 | 0.16 | 0.21 | 0.15 | 0.33 | 0.25 | N.P. |
| | RRT 1.16 | 0.07 | 0.13 | 0.24 | 0.19 | 0.51 | 0.18 | |
| | RRT 1.54 | <0.05 | <0.05 | 0.23 | 0.09 | 0.27 | 0.15 | |
| 1:1.5 | RRT 1.07 | 0.10 | 0.13 | 0.11 | 0.15 | 0.20 | 0.13 | 0.18 |
| | RRT 1.16 | 0.05 | 0.08 | 0.10 | 0.09 | 0.14 | 0.09 | 0.15 |
| | RRT 1.54 | <0.05 | <0.05 | 0.15 | 0.11 | 0.26 | 0.17 | 0.26 |
| 1:2 | RRT 1.07 | 0.07 | 0.14 | 0.12 | 0.16 | 0.15 | N.P. | N.P. |
| | RRT 1.16 | 0.06 | 0.08 | 0.09 | 0.08 | 0.10 | | |
| | RRT 1.54 | <0.05 | <0.05 | 0.13 | <0.05 | 0.26 | | |

Solid state form - XRPD

| | | | | | | | |
|---|---|---|---|---|---|---|---|
| 1:1 | am | am | N.P. | am | am | not am | N.P. |
| 1:1.5 | am | am | N.P. | am | N.P. | am | am |
| 1:2 | am | am | N.P. | am | N.P. | am | am |

¹HDPE bottle with 2 g desiccant
²Alu/Alu blister
N.P.: not performed
am: amorphous

### Example 3: Stability Results for 50 mg Capsules (Sunitinib L-Malate:PVP 1:1.5) Stored in Several Type of Blister Pack Materials with Different Water Vapour Transmission Rates (WVTR), Determined at 23° C./100% RH

The capsules have the composition as given in example 1 and are prepared by the process given in that example.

| | | 40° C./75% RH | | |
|---|---|---|---|---|
| | | 3 months | | |
| | | Triplex | Aclar 3000 | Alu/Alu |
| | | WVTR: 0.15 | WVTR: 0.07 | WVTR: ≤0.01 |
| Impurities | t = 0 | g/m² × d | g/m² × d | g/m² × d |

Main degradation impurities - UHPLC (%)

| | | | | |
|---|---|---|---|---|
| RRT 1.07 | <0.05 | 0.37 | 0.26 | 0.13 |
| RRT 1.16 | <0.05 | 0.46 | 0.29 | 0.11 |
| RRT 1.54 | <0.05 | 0.34 | 0.32 | 0.19 |

### Example 4: Stability Results for 50 mg Capsules (Sunitinib L-Malate:PVP 1:1.5) without and with Different Type of Antioxidants

The capsules have the composition as given in example 1 and are prepared by the process given in that example. The antioxidants are added intragranularly.

| Antioxidant, | | | 40° C./75% RH | | | |
|---|---|---|---|---|---|---|
| 0.5% | Impuri- | | 1 month | | 3 months | |
| intragranular | ties | t = 0 | Bottle¹ | Blister² | Bottle¹ | Blister² |

Main degradation impurities - UHPLC (%)

| | | | | | | |
|---|---|---|---|---|---|---|
| No | RRT 1.07 | 0.10 | 0.13 | 0.11 | 0.15 | 0.20 |
| antioxidant | RRT 1.16 | 0.05 | 0.08 | 0.10 | 0.09 | 0.14 |
| | RRT 1.54 | <0.05 | <0.05 | 0.15 | 0.11 | 0.26 |
| Vitamin C | RRT 1.07 | 0.05 | 0.33 | 0.40 | 0.42 | N.P. |
| | RRT 1.16 | <0.05 | 0.12 | 0.31 | 0.14 | |
| | RRT 1.54 | <0.05 | <0.05 | 0.09 | 0.07 | |
| Vitamin E | RRT 1.07 | 0.08 | N.P. | N.P. | 0.17 | 0.22 |
| | RRT 1.16 | 0.08 | | | 0.12 | 0.16 |
| | RRT 1.54 | <0.05 | | | 0.11 | 0.35 |
| BHT | RRT 1.07 | <0.05 | N.P. | N.P. | 0.18 | 0.25 |
| | RRT 1.16 | 0.06 | | | 0.10 | 0.15 |
| | RRT 1.54 | <0.05 | | | 0.10 | 0.30 |

¹HDPE bottle with 2 g desiccant
²Alu/Alu blister
N.P.: not performed

### Example 5: Stability Results for 50 mg Capsules (Sunitinib L-Malate:PVP 1:1.5) Comprising Different Grades (Peroxide Content) of PVP

The capsules have the composition as given in example 1 and are prepared by the process given in that example.

| | | | 40° C./75% RH | | | |
|---|---|---|---|---|---|---|
| | Impuri- | | 1 month | | 3 months | |
| PVP grade | ties | t =0 | Bottle¹ | Blister² | Bottle¹ | Blister² |

Main degradation impurities - UHPLC (%)

| | | | | | | |
|---|---|---|---|---|---|---|
| Plasdone ™ | RRT 1.07 | 0.10 | 0.13 | 0.11 | 0.15 | 0.20 |
| K29/32 | RRT 1.16 | 0.05 | 0.08 | 0.10 | 0.09 | 0.14 |
| (<400 ppm | RRT 1.54 | <0.05 | <0.05 | 0.15 | 0.11 | 0.26 |
| peroxides) | | | | | | |
| Kollidon ® | RRT 1.07 | <0.05 | N.P. | N.P. | 0.07 | 0.13 |
| 30 LP | RRT 1.16 | <0.05 | | | 0.05 | 0.11 |
| (<150 ppm | RRT 1.54 | <0.05 | | | 0.08 | 0.19 |
| peroxides) | | | | | | |

¹HDPE bottle with 2 g desiccant
²Alu/Alu blister
N.P.: not performed

### Example 6: Stability Results for 50 mg Capsules (Sunitinib L-Malate:PVP 1:1.5) with Different Type of Capsule Shells

The capsules have the composition as given in example 1 and are prepared by the process given in that example.

| | | | 40° C./75% RH | |
|---|---|---|---|---|
| | | | 1 month | |
| | | | HDPE bottle | |
| | | | with 2 g | Alu/Alu |
| Capsule shell | Impurities | t = 0 | desiccant | blister |

Main degradation impurities - UHPLC (%)

| | | | | |
|---|---|---|---|---|
| Gelatin, colored | RRT 1.07 | <0.05 | <0.05 | 0.05 |
| LOD: 13-16%; containing | RRT 1.16 | <0.05 | <0.05 | 0.06 |
| several metal oxides | RRT 1.54 | <0.05 | 0.07 | 0.20 |
| HPMC, transparent | RRT 1.07 | <0.05 | <0.05 | 0.07 |
| LOD: ≤9%; free of | RRT 1.16 | <0.05 | <0.05 | 0.10 |
| metal oxides | RRT 1.54 | <0.05 | 0.07 | 0.25 |

### Example 7: Stability Results for 50 mg Capsules (Sunitinib L-Malate:PVP 1:1.5) Stored in Different Sizes of Bottles (Headspace Volume)

The capsules have the composition as given in example 1 and are prepared by the process given in that example. The PVP used is Kollidon® 30 LP

| | | | |
|---|---|---|---|
| HDPE bottle, | | | |
| with desiccant | | | 40° C./75% RH |
| (2 g) | Impurities | t = 0 | 6 months |

Main degradation impurities - UHPLC (%)

| | | | |
|---|---|---|---|
| 75 ml/18 cap | RRT 1.07 | <0.05 | 0.09 |
| | RRT 1.16 | <0.05 | 0.10 |
| | RRT 1.54 | <0.05 | 0.17 |
| 30 ml/18 cap | RRT 1.07 | <0.05 | 0.07 |
| | RRT 1.16 | <0.05 | 0.09 |
| | RRT 1.54 | <0.05 | 0.12 |

¹ HDPE bottle with 2 g desiccant
² Alu/Alu blister
N.P.: not performed

### Example 8: Stability Results for Slugs (Sunitinib L-Malate:PVP 1:1.5) Comprising Different Type of Lubricants

The slugs have the composition as given in example 1 and are prepared by the process given in that example. Magnesium stearate is in two of the compositions replaced by other lubricants.

50° C.

| | | | | |
|---|---|---|---|---|
| | | | 2 weeks | 4 weeks |
| | | | Alu/Alu | Alu/Alu |
| Lubricant | Impurities | t = 0 | blister | blister |

Main degradation impurities - UHPLC (%)

| | | | | |
|---|---|---|---|---|
| Magnesium | RRT 1.07 | 0.05 | 0.22 | 0.18 |
| stearate | RRT 1.16 | <0.05 | 0.22 | 0.33 |
| | RRT 1.54 | <0.05 | 0.24 | 0.28 |
| Sodium stearyl | RRT 1.07 | <0.05 | 0.22 | 0.19 |
| fumarate | RRT 1.16 | <0.05 | 0.21 | 0.32 |
| | RRT 1.54 | <0.05 | 0.28 | 0.32 |
| Stearic acid | RRT 1.07 | 0.05 | 0.23 | 0.21 |
| | RRT 1.16 | <0.05 | 0.24 | 0.32 |
| | RRT 1.54 | <0.05 | 0.26 | 0.26 |

### Example 9: Stability Results for 50 mg Capsules (Sunitinib L-Malate:PVP 1:1.5) Stored in Different Type of Alu/Alu Blister Pack Materials (with and without Desiccant)

The capsules have the composition as given in example 1 and are prepared by the process given in that example.

| | | 40° C./75% RH | | | | | |
|---|---|---|---|---|---|---|---|
| | | 1 month | | 2 months | | 3 months | |
| | | Alu/ | Dessi- | Alu/ | Dessi- | Alu/ | Dessi- |
| Impurities | t = 0 | Alu | flex ™ | Alu | flex ™ | Alu | flex ™ |

Main degradation impurities - UHPLC (%)

| | | | | | | | |
|---|---|---|---|---|---|---|---|
| RRT 1.07 | <0.05 | 0.07 | <0.05 | 0.14 | <0.05 | 0.19 | 0.05 |
| RRT 1.16 | <0.05 | 0.15 | <0.05 | 0.19 | <0.05 | 0.25 | 0.05 |
| RRT 1.54 | <0.05 | 0.20 | <0.05 | 0.24 | 0.06 | 0.27 | 0.10 |

Dessiflex™ Ultra: dessicated alu/alu blister having a water absorption capacity of at least 6.1 g/m² at 40° C./90% RH. The water absorption capacity of the blister is determined by gravimetric measurement. A 100 cm² sample is exposed in a climate chamber (40° C./90% RH) for 24 hr. The weight increase is the humidity absorption capacity per 100 cm² which can be expressed (multiplying with 100) into water absorption capacity in g/m².

### Example 10: Stability Results for 50 mg Capsules (Sunitinib L-Malate:PVP 1:1.5) Stored in Capped HDPE Bottles with Different Amounts of Desiccant (2 g, 6 g) or Oxygen Scavenger (1 g, 2.15 g) in the Caps

The capsules have the composition as given in example 1 and are prepared by the process given in that example.

| | | 40° C/.75% RH | | | | | |
|---|---|---|---|---|---|---|---|
| | | 1 month | | | 3 months | | |
| | | | | HDPE | | | HDPE |
| | | HDPE | HDPE | 1 g O₂ | HDPE | HDPE | 2.15 g O₂ |
| Impurities | t = 0 | 2 g des.¹ | 6 g des.² | scav. | 2 g des.¹ | 6 g des.² | scav. |

Main degradation impurities - UHPLC (%)

| | | | | | | | |
|---|---|---|---|---|---|---|---|
| RRT 1.07 | <0.05 | <0.05 | <0.05 | <0.05 | 0.08 | <0.05 | 0.06 |
| RRT 1.16 | <0.05 | <0.05 | <0.05 | 0.05 | 0.07 | <0.05 | 0.06 |
| RRT 1.54 | <0.05 | 0.05 | <0.05 | 0.16 | 0.10 | <0.05 | 0.22 |

¹Water absorption capacity: 5.3 g/dm³ at 23° C./40% RH
²Water absorption capacity: 16 g/dm³ at 23° C./40% RH

1. A pharmaceutical composition comprising a solid dispersion of sunitinib L-malate and polyvinylpyrrolidone in a primary packaging comprising means to absorb water.

2. The composition according to claim 1, wherein the primary packaging is a blister pack having a water absorption capacity of at least 2.0 g/m² at 40° C./90% RH.

3. The composition according to claim 2, wherein the blister pack is a laminated cold forming blister pack comprising desiccant.

4. The composition according to claim 1, wherein the primary packaging is a capped bottle having a water absorption capacity of at least 5.3 g/dm³ at 23° C./40% RH.

5. The composition according to claim 4, wherein the capped bottle is a HDPE bottle and the cap comprises desiccant.

6. The composition according to claim 1, wherein sunitinib L-malate is present in amorphous form.

7. The composition according to claim 1, wherein the ratio of sunitinib L-malate to polyvinylpyrrolidone ranges from 1:1 to 1:2.

8. The composition according to claim 1 being a hard shell capsule.

9. The composition according to claim 1, further comprising one or more pharmaceutically acceptable excipients chosen from one or more diluents, disintegrants, or lubricants.

10. The composition according to claim 1, wherein microcrystalline cellulose is present as intragranular excipient.

11. The composition according to claim 9, wherein microcrystalline cellulose, croscarmellose sodium and magnesium stearate are present as extragranular excipients.

12. A process to prepare the pharmaceutical composition according to claim 1, which comprises wet granulating sunitinib L-malate and polyvinylpyrrolidone to form said solid dispersion.

13. The process according to claim 12, wherein the granulation step is performed in an aqueous acidic solvent system.

14. The process according to claim 12, wherein the granulation is performed by spraying a solution of sunitinib L-malate and polyvinylpyrrolidone in an aqueous acidic solvent system over microcrystalline cellulose followed by evaporation of the solvent to form a granulate containing said solid dispersion.

15. (canceled)

16. The composition according to claim 1, wherein said composition comprises granules that contain said solid dispersion and at least one extragranular excipient selected from the group consisting of diluents, disintegrants, and lubricants.

17. The composition according to claim 16, wherein said granules further comprise microcrystalline cellulose and said extragranular excipient comprises microcrystalline cellulose, croscarmellose sodium and magnesium stearate.

18. The composition according to claim 17, wherein the ratio of sunitinib L-malate to polyvinylpyrrolidone ranges from 1:1 to 1:2.

19. The composition according to claim 18, wherein said polyvinylpyrrolidone has a molecular weight ranging from 30,000 to 60,000.

20. The process according to claim 14, which further comprises:
blending said granulate with one or more additional excipients to form a final blend;
filling said final blend into capsules or compressing into tablets; and
packing said capsules or tablets into said primary packaging.
